(19) United States
(12) Patent Application Publication
(10) Pub. No.: US 2020/0384053 A1
(43) Pub. Date: Dec. 10, 2020

(54) Ophthalmic Compositions and Medical Uses Thereof in Treatment of Myopia

(71) Applicant: SunDragon Bio-medical Technology Co., Taipei City (TW)

(72) Inventor: Lei Wan, Taichung City (TW)

(21) Appl. No.: 16/431,248

(22) Filed: Jun. 4, 2019

(51) Int. Cl. A61K 36/704; A61K 36/536; A61K 9/00; A61P 27/02

(52) U.S. Cl. CPC A61K 36/704; A61P 27/02; A61K 9/0048; A61K 36/536

(57) ABSTRACT

The invention relates to ophthalmic compositions containing an aqueous extract derived from Polygonum cuspidatum Sieb. et Zucc. and/or an aqueous extract derived from Prunella vulgaris. The invention further relates to the use of the ophthalmic compositions in the treatment of myopia.

### FIELD OF THE INVENTION

The invention relates to ophthalmic compositions containing an herbal plant extract, and more particularly, to ophthalmic compositions containing an aqueous extract derived from the botanical species Polygonum cuspidatum Sieb. et Zucc., Prunella vulgaris or a combination thereof. The invention further relates to the use of the ophthalmic compositions in the treatment of myopia.

### DESCRIPTION OF THE RELATED ART

The prevalence of myopia has rapidly increased in recent decades and has led to a considerable global public health concern. Globally, there are approximately 153 million people over the age of 5 years who suffer from visual defects, 8 million of whom suffer from blindness because of uncorrected myopia and other refractive errors (REs). In the United States alone, the economic costs of myopia have increased to US$250 million per year. Myopia is a prominent and often undertreated eye disease. Although most cases of myopia can be corrected with glasses, contact lenses, or refractive surgery, uncorrected REs still account for approximately 33% of visual impairments. High-degree myopia is a particularly dangerous visual affliction because of the higher risks of macular and retinal complications. Myopia results primarily from abnormal elongation of the vitreous chamber of the eye. This condition is recapitulated in the monocular form deprivation (MFD) animal model, which has been used to study myopia pathogenesis. Eye elongation is associated with remodeling of the sclera, loss of scleral tissue through reduced connective tissue synthesis, and increased collagen I (COL1) degradation, resulting in changes in the composition and ductility of the sclera. Recent studies in monkeys have demonstrated that the retina, specifically, photoreceptors and retinal pigment epithelium (RPE), plays a crucial role in modulating eye growth and axial length through producing activating signals that promote scleral tissue remodeling.

Animal studies of myopia have shown that atropine, a nonselective muscarinic acetylcholine receptor (mAChR) antagonist, effectively prevents axial elongation, which leads to myopia. Atropine inhibits myopia progression in tree shrews, monkeys, chickens, guinea pigs, rats, mice, and Syrian hamsters, and its effectiveness has also been demonstrated in human clinical trials. However, the mechanism of this effect remains to be investigated.

Various molecules have been implicated in myopia progression. In myopic eyes, transforming growth factor-β (TGF-β) and matrix metalloproteinase 2 (MMP2) expression is elevated, whereas COL1 expression is down-regulated. It has been shown that TGF-β regulates cellular functions such as cell growth, differentiation, inflammation, and wound healing, whereas MMP family members play major roles in the breakdown of the extracellular matrix, tissue reconstruction, and vascularization during the inflammatory response. The dysregulation of MMPs has been proposed as a mechanism of pathogenesis in myopic eyes; MMP2 expression is upregulated in the sclera of chickens and tree shrews in which myopia has been induced through form deprivation. TGF-β regulates the level of MMP2 through activation of nuclear factor-κB (NF-κB), a transcription factor that modulates the expression of various inflammatory cytokines in fibroblasts.

The role of inflammation in myopia progression has been investigated (Lin H. J. et al. EBioMedicine 2016; 10: 269-281). Uveitis can induce acute or constitutive myopia and myopic shift, and acute myopia has been observed in patients with acute scleritis. A 26-year follow-up of patients with juvenile chronic arthritis (JCA) revealed myopic REs in a greater proportion of these patients than in age-matched control patients, suggesting a correlation between JCA and myopia. The same study suggested that the higher incidence of myopia was due to the weakening of scleral connective tissue as a result of chronic inflammation. In addition, acute-onset myopia may be a presenting feature of systemic lupus erythematosus (SLE). The high incidence of uveitis among JCA patients may increase the progression of myopia. Higher expression levels of the inflammatory cytokines IL-6 and TNF-α were found in the aqueous humor of uveitis patients. These increased levels of IL-6 and TNF-α in the eye support the progression of myopia. The systemic acute or chronic inflammatory state associated with these diseases likely increases the occurrence of myopia.

U.S. Patent Publication No. 2016/0120833, assigned to the Applicant, discloses a therapeutic method for treating and/or relieving myopia, which involves topical application of an anti-inflammatory agent to patient's eyes. The patent publication proposes a direct linkage from inflammatory cascade to myopia progression by showing that the expression of inflammatory cytokines, including IL-6, IL-1β, TGF-β and TNF-α, was up-regulated at both protein and RNA levels in a monocular form deprivation (MFD)-induced myopia animal model, and it can be suppressed by administering an anti-inflammatory agent, such as atropine, resveratrol, diacerein and diclofenac.

Tiger cane (Polygonum cuspidatum Sieb. et Zucc., synonym Fallopia japonica, or Reynoutria japonica Houtt), also known as Japanese Knotweed, Japanese bamboo and Huzhang in mandarin, is a traditional and popular Chinese medicinal herb. The underground part of this herb has been used as medicine for thousands of years, mainly removing poisonous substances and relieving cough, joint pain, scald, jaundice, hepatitis, amenorrhea and snake bites. Some chemical constituents derived from P. cuspidatum were found bioactive and useful in the treatment of inflammation, favus, chronic bronchitis and hyperlipemia (for review, see Zhang H. et al., Evid. Based Complement. Alternat. Med. Volume 2013, Article ID 208349; and Peng W. et al., J. Ethnopharmacol. 2013 Jul. 30, 148(3):729-45). Traditionally, underground rhizomes of tiger cane are dug out from soil in spring and autumn and dried under the sun after chopped into segments or thick slices. The dried rhizome slices are commercially available from traditional herbal medicine stores.

Prunella vulgaris is another herbal plant commonly found in Europe and Asia, also known as common self-heal, heal-all, woundwort, heart-of-the-earth, carpenter's herb, brownwort, blue curls, and Xia Ku Cao in mandarin. The plant in whole is edible, and the above-ground parts of the herb can be powdered and brewed to make herbal beverage. The herb has a long history of medicinal use, and traditionally the leaves are applied to wounds to promote healing. A decoction of the leaf parts of the herb is useful in treating fevers, diarrhea, sore mouth and throat, and internal bleeding.

Extracts of P. cuspidatum and P. vulgaris have been shown to exhibit beneficial medicinal properties and have a potential of being an effective alternative remedy for treatment of a variety of disease conditions. Taiwanese Patent No. 1304342 teaches aquatic, acetonic and ethanolic extracts of P. cuspidatum, which showed antimicrobial effects against enterovirus in vitro. U.S. Pat. No. 5,837,257 teaches a hot water extract of P. vulgaris exhibiting anti-viral activity against HIV in vitro. Collins N. H. et al. demonstrated that a methanol extract of P. vulgaris presented anti-estrogenic activity in an endometrial xenograft mouse model, suggesting the usefulness of the herb as a complementary alternative medicine for the treatment of estrogen-dependent disorders in female patients, such as endometriosis and breast and uterine cancers (Collins N. H. et al., Biol. Reprod. 2009, 80, 375-383). Chinese Patent Publication No. 101284048A discloses an ophthalmic preparation in which P. vulgaris is included, among other herbal ingredients. The preparation is alleged to have broad spectrum uses in treating different kinds of ophthalmic disorders, including myopia, asthenopia, cataract and retinopathy. Chinese Patent Publication Nos. 104288317A and 103719375A disclose medicinal preparations derived from P. cuspidatum and P. vulgaris, in combination with a tremendous variety of other herbal medicines.

To the best of the inventors' knowledge, the individual and combined effects of P. cuspidatum and P. vulgaris on myopia progression remain unknown in the art.

### SUMMARY OF THE INVENTION

Retinal pigment epithelium, or abbreviated herein as RPE, is the pigmented cell layer sandwiched between the underlying choroid and overlying retinal visual cells and is a vital tissue for the maintenance of photoreceptor function and environment of outer retina. It is known that the dysfunction of RPE is related to age-related macular degeneration and retinitis pigmentosa and also involves in diabetic retinopathy and myopia progression (Lin H. J. et al. Supra). According to the preliminary experiments disclosed herein, exogenous application of inflammatory cytokines, i.e., IL-6 and TNF-α, to RPE cells was found to induce endogenous expression of IL-6, IL-8 and TNF-α. The cytokine-induced inflammation model thus established was used to test herb extracts for anti-inflammatory activity in vitro.

The present invention is primarily based on the unexpected findings that P. cuspidatum alone, P. vulgaris alone, and a combination thereof, especially the aqueous extracts thereof, are effective in inhibiting the endogenous expression of inflammatory cytokines in RPE cells, indicating that the herbs possess anti-inflammatory activity, and further suggesting their medicinal potential in the treatment of myopia. The present inventors further surprisingly found that the administration of a combined extract of P. cuspidatum with P. vulgaris resulted in significantly higher inhibitory effects on the cytokine expression as compared with individual herb extracts acting alone, indicating synergistic actions of the two herbs. Consistent results were obtained in vivo using monocular form deprivation (MFD) animal models, showing that the individual or combined treatment of P. cuspidatum and P. vulgaris reduced the change in axial length in the MFD eyes and also reduced the expression of inflammatory cytokines in the MFD eyes, as compared to the results obtained in the control group, suggesting that administration of the herb extracts inhibited myopia progression. In particular, the inhibitory effects of the herb extracts on myopia progression were found comparable to a known anti-inflammatory agent, atropine. Taken together, the findings herein suggest that P. cuspidatum and P. vulgaris, either alone or in combination, are therapeutically useful in the treatment of myopia.

Accordingly, one object of the invention is to provide an ophthalmic composition comprising a therapeutically effective amount of an aqueous extract derived from a botanical species selected from the group consisting of Polygonum cuspidatum Sieb. et Zucc., Prunella vulgaris, and a combination thereof, in combination with an ophthalmically acceptable carrier.

Another object of the invention is to provide a method for treating myopia in a subject, comprising administering to said subject an ophthalmic composition comprising a therapeutically effective amount of an aqueous extract derived from a botanical species selected from the group consisting of Polygonum cuspidatum Sieb. et Zucc., Prunella vulgaris, and a combination thereof, in combination with an ophthalmically acceptable carrier.

Specifically, in the first aspect provided herein is an ophthalmic composition comprising, preferably consisting essentially of, and more preferably consisting of, a therapeutically effective amount of an aqueous extract derived from Polygonum cuspidatum Sieb. et Zucc., and an ophthalmically acceptable carrier.

In the second aspect provided herein is use of an ophthalmic composition in the manufacture of a medicament for treating myopia in a subject comprising, preferably consisting essentially of, and more preferably consisting of, a therapeutically effective amount of an aqueous extract derived from Polygonum cuspidatum Sieb. et Zucc., and an ophthalmically acceptable carrier.

In the third aspect provided herein is a method for treating myopia in a subject, comprising administering to said subject an ophthalmic composition comprising, preferably consisting essentially of, and more preferably consisting of, a therapeutically effective amount of an aqueous extract derived from Polygonum cuspidatum Sieb. et Zucc., and an ophthalmically acceptable carrier.

In the fourth aspect provided herein is an ophthalmic composition comprising, preferably consisting essentially of, and more preferably consisting of, a therapeutically effective amount of an aqueous extract derived from Prunella vulgaris, and an ophthalmically acceptable carrier.

In the fifth aspect provided herein is use of an ophthalmic composition in the manufacture of a medicament for treating myopia in a subject comprising, preferably consisting essentially of, and more preferably consisting of, a therapeutically effective amount of an aqueous extract derived from Prunella vulgaris, and an ophthalmically acceptable carrier.

In the sixth aspect provided herein is a method for treating myopia in a subject, comprising administering to said subject an ophthalmic composition comprising, preferably consisting essentially of, and more preferably consisting of, a therapeutically effective amount of an aqueous extract derived from Prunella vulgaris, and an ophthalmically acceptable carrier.

In the seventh aspect provided herein is a composition consisting essentially of, and preferably consisting of, a therapeutically effective amount of an aqueous extract derived from Polygonum cuspidatum Sieb. et Zucc., in combination with a therapeutically effective amount of an aqueous extract derived from Prunella vulgaris.

In the eighth aspect provided herein is an ophthalmic composition comprising, preferably consisting essentially of, and more preferably consisting of, a therapeutically effective amount of an aqueous extract derived from Polygonum cuspidatum Sieb. et Zucc., in combination with a therapeutically effective amount of an aqueous extract derived from Prunella vulgaris, and an ophthalmically acceptable carrier.

In the ninth aspect provided herein is use of an ophthalmic composition in the manufacture of a medicament for treating myopia in a subject comprising, preferably consisting essentially of, and more preferably consisting of, a therapeutically effective amount of an aqueous extract derived from Polygonum cuspidatum Sieb. et Zucc., in combination with a therapeutically effective amount of an aqueous extract derived from Prunella vulgaris, and an ophthalmically acceptable carrier.

In the tenth aspect provided herein is a method for treating myopia in a subject, comprising administering to said subject an ophthalmic composition comprising, preferably consisting essentially of, and more preferably consisting of, a therapeutically effective amount of an aqueous extract derived from Polygonum cuspidatum Sieb. et Zucc., in combination with a therapeutically effective amount of an aqueous extract derived from Prunella vulgaris, and an ophthalmically acceptable carrier.

In the preferred embodiments, the aqueous extracts are obtained using an aqueous solvent selected from water, C₁₋₆ alkanols, C₁₋₄ alkylene glycols, aqueous carbohydrate solutions, salt solutions thereof, and mixtures thereof. In the more preferred embodiments, the aqueous solvent is selected from water and salt solutions thereof.

In a preferred embodiment, the ophthalmic composition is formulated into a dosage form selected from the group consisting of an eye drop, an eye wash, an eye spray, an ointment, a cream and a gel.

In the preferred embodiments, the ophthamically acceptable carrier is selected from the group consisting of polyoxyethylene castor oil ether (Cremophor EL), alkyl dimethyl benzyl ammonium (BKC), lecithin, cholesterol, phosphate-buffered saline (PBS), cyclodextrin, tween 80, castor oil, artificial tears, and a combination thereof. In the more preferred embodiments, the ophthalmically acceptable carrier comprises artificial tears.

In the preferred embodiments, the subject is selected from the group consisting of human and non-human vertebrates. In the more preferred embodiments, the subject is human.

In the preferred embodiments, the ophthalmic composition is administered at an amount ranging from 0.01 ng/kg body weight/day to 100 ng/kg body weight/day. In the more preferred embodiments, the ophthalmic composition is administered at an amount ranging from 0.1 ng/kg body weight/day to 10 ng/kg body weight/day.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The above and other objects, features and effects of the invention will become apparent with reference to the following description of the preferred embodiments taken in conjunction with the accompanying drawings, in which:

FIGS. 1A-1C are histograms showing that endogenous expression of IL-6 (FIG. 1A), IL-8 (FIG. 1B) and TNF-α (FIG. 1C) in ARPE-19 cells was induced by exogenous administration of IL-6 and TNF-α;

FIGS. 2A-2C are histograms showing that IL-6 and TNF-α worked synergistically to induce the endogenous expression of IL-6 (FIG. 2A), IL-8 (FIG. 2B) and TNF-α (FIG. 2C) in ARPE-19 cells;

FIGS. 3A-3C are another histograms showing that IL-6 and TNF-α worked synergistically to induce the endogenous expression of IL-6 (FIG. 3A), IL-8 (FIG. 3B) and TNF-α (FIG. 3C) in ARPE-19 cells;

FIG. 4 is a histogram showing that the expressed levels of IL-6 over time after removal of IL-6 and TNF-α from the culture media;

FIGS. 5A-5B are histograms showing that the administration of the aqueous herb extracts A and C at a concentration from 10 to 120 μg/ml exhibited no toxicity to ARPE-19 cells;

FIG. 6 is a histogram showing that the cytokine-induced expression of IL-6 in ARPE-19 cells was suppressed by the herb Extract A and Extract C;

FIGS. 7A-7C are histograms showing that the cytokine-induced expression of IL-6, IL-8 and TNF-α in ARPE-19 cells was suppressed by the herb Extract A in combination with the herb Extract C in a dose-dependent manner;

FIGS. 8A-8C are histograms showing that the cytokine-induced expression of IL-6, IL-8 and TNF-α in human RPE primary cells was suppressed by the herb Extract A in combination with the herb Extract C in a dose-dependent manner;

FIGS. 9A-9B are histograms showing the change in axial length of the non-MFD eyes (FIG. 9A) and the MFD eyes (FIG. 9B) in the Control and treated groups; and

FIGS. 10A-10F are histological images showing the immunohistochemical analysis of TNF-α, IL-6, IL-8, NFκB, MMP2 and CD55 expression in the MFD eyes in the Control and treated groups.

### DETAILED DESCRIPTION OF THE INVENTION

Unless specified otherwise, the following terms as used in the specification and appended claims are given the following definitions. It should be noted that the indefinite article “a” or “an” as used in the specification and claims is intended to mean one or more than one, such as “at least one,” “at least two,” or “at least three,” and does not merely refer to a singular one. In addition, the terms “comprising/comprises,” “including/includes” and “having/has” as used in the claims are open languages and do not exclude unrecited elements. The term “or” generally covers “and/or”, unless otherwise specified. The term “about” or “substantially” used throughout the specification and appended claims are used to describe and account for slight changes that do not materially affect the nature of the invention.

As used herein, the term “ophthalmic composition” refers to a composition for topical ocular administration.

The term “Polygonum cuspidatum Sieb. et Zucc.” or abbreviated as “P. cuspidatum” is used herein to encompass the newly harvested, unprocessed or processed whole plant and plant parts of the botanical species, especially the underground part thereof, such as the dried rhizomes and roots thereof commercially available from the traditional herbal medicine distributors.

The term “Prunella vulgaris” or abbreviated as “P. vulgaris” as used herein refers to the newly harvested, unprocessed or processed whole plant and plant parts of the botanical species, especially the above-ground parts thereof, such as the dried whole plant and the dried ears thereof commercially available from the traditional herbal medicine distributors.

The term “aqueous extract,” as used herein, may refer to a composition prepared by contacting plant material with an aqueous solvent following standard extraction procedures. The term “derived from” as used herein is intended to indicate the source of the plant material from which the aqueous extract is prepared. Non-limiting examples of the aqueous solvent include water, C₁₋₆ alkanols, C₁₋₄ alkylene glycols, aqueous carbohydrate solutions, salt solutions thereof, and mixtures thereof. Desirably, the aqueous solvent is selected from water and salt solutions thereof. The plant material may be ground and the pressure and temperature may be elevated for enhancing the extraction efficiency. In a preferred embodiment, the aqueous extract is obtained by extraction with deionized water at a high temperature, such as in an autoclave at 121° C. under a pressure of 1.2 kg/cm². The duration of extraction depends on the yield of extraction and normally lasts 30 minutes to one day, preferably 1-12 hours, such as 1-3 hours. The term “aqueous extract” encompasses crude extracts, prepared by a simple aqueous extraction, as well as crude extracts that have been subjected to one or more separation and/or purification steps, including substantially purified and purified active ingredient(s) and concentrates or fractions derived from a crude extract by subjecting the crude extract to one or more additional extraction, concentration, fractionation, filtration, condensation, distillation or other purification step. The extract may be in liquid form, such as a solution, concentrate or distillate, or in semi-liquid form, such as a gel. The aqueous extract can, if appropriate and desired, be lyophilized and stored in a sterile ampoule ready for reconstitution by the addition of an aqueous solvent, such as sterile water.

The term “ophthalmically acceptable carrier” refers to an agent or medium which does not interfere with the effectiveness of the aqueous extracts described herein and which is not excessively toxic to the host at the concentration at which it is administered. The term includes but is not limited to solvents, dispersion media, antibacterial and antifungal agents, isotonic agents, adsorption delaying agents, penetration enhancers, pH-adjusting agents, thickening agents, antioxidants, chelating agents, and the like. The use of the ophthalmically acceptable carrier is well known in the art (see, for example, Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999)).

In one preferred embodiment, the ophthamically acceptable carrier is selected from the group consisting of polyoxyethylene castor oil ether (Cremophor EL), alkyl dimethyl benzyl ammonium (BKC), lecithin, cholesterol, phosphate-buffered saline (PBS), cyclodextrin, tween 80, castor oil, artificial tears, and a combination thereof. In a more preferred embodiment, the ophthamically acceptable carrier comprises artificial tears. In the context of the invention, the term “artificial tears” refers to non-irritating lubricant eye drops used to replace the function of natural tears. The ingredients in artificial tears include but are not limited to water, salts, sodium hyaluronate, carboxymethyl cellulose, hydroxypropyl methyl cellulose or hydroxypropyl cellulose prime.

The aqueous extracts herein can be readily formulated with or prepared with the ophthalmically acceptable carrier. In some embodiments, the term may encompass excipients and auxiliaries which facilitate processing of the aqueous extracts herein into compositions which can be applied ophthalmically. The ophthalmic compositions herein may be prepared by various techniques. Such techniques include bringing into association the aqueous extracts with an appropriate carrier. The ophthalmic compositions may be formulated in the form of aqueous solutions, aqueous suspensions, oil emulsions, water-in-oil emulsions, water-in-oil-in-water emulsions, creams, gels and other forms suitable for ophthalmic delivery. In one embodiment, the ophthalmic compositions are formulated into a non-invasive liquid or semi-liquid dosage form selected from the group consisting of an eye drop, an eye wash, an eye spray, an ointment, a cream and a gel. In another embodiment, the compositions are prepared as injectables, either as liquid solutions or suspensions. Solid forms suitable for solution in, or suspension in, liquid prior to injection may alternatively be prepared.

The term “comprise” or “comprising”, when used in a claim, means the elements recited, or their equivalents in structure or function, plus any other element or elements which are not recited. The transitional phrase “consist of” or “consisting of” is closed, and excludes all additional elements. The phrase “consist essentially of” or “consisting essentially of”, with respect to the constitutive elements of the ophthalmic composition defined in the claims, means that the composition contains the indicated elements and may contain additional elements only if the additional elements do not materially alter the basic and novel characteristics of the invention as a composition for treatment of myopia. Preferably, such additives are not present at all or only in trace amounts. For instance, a claimed composition consisting essentially of an aqueous extract derived from an herb and an ophthalmically acceptable carrier would not exclude trace contaminants from the preparation steps of the extract and substances, such as phosphate-buffered saline (PBS), preservatives and salts, which do not materially affect the anti-inflammation property of the composition, as well as the suppressive property of the composition on myopia progression.

In one aspect, the invention contemplates the use of the ophthalmic compositions disclosed herein in the manufacture of a medicament for treating myopia in a subject, as well as therapeutic methods for treating myopia in a subject by administering the ophthalmic compositions herein to the subject.

The term “myopia”, also known as nearsightedness, refers to a condition associated with a refractive error (RE) of one or more eyes, a problem with focusing light accurately onto the retina due to an overly long axial length of the eye ball. The term may encompass a variety of levels (mild myopia, from 0 to −3 diopters; moderate myopia, from −3 to −5 diopters; and high myopia, from −5 or lower), and types and subtypes of myopia of various etiologies and causes, either known or unknown, including, but not limited to, simple myopia, degenerative myopia, and form-deprivation myopia.

The term “treating” or “treatment” includes prevention of the initiation of myopia, alleviation of myopia after its emergence, reduction of the severity/intensity of myopia, amelioration of one or more of the symptoms caused by or leading to myopia, or deceleration or inhibition of the advancement of myopia progression and/or myopia shift index.

As used herein, the term “subject” is intended to encompass human or non-human vertebrates, such as non-human mammal. Non-human mammals include livestock animals, companion animals, laboratory animals, and non-human primates. Non-human subjects also include, without limitation, horses, cows, pigs, goats, dogs, cats, mice, rats, guinea pigs, gerbils, hamsters, mink, rabbits and fish. It is understood that the preferred subject is a human, especially a human patient afflicted with or at risk for myopia.

For the purpose of research, the term “subject” may refer to a biological sample as defined herein, which includes but is not limited to a cell, tissue, or organ. Accordingly, the invention disclosed herein is intended to be applied in vivo as well as in vitro.

According to the invention, the term “administering” includes dispensing, delivering or applying the ophthalmic composition to a subject. In one embodiment, the composition is administered to a subject before, during and/or after the onset of myopia. The ophthalmic composition can be applied topically or by other techniques, known to persons skilled in the art, such as injection to the eye or its related tissues. Examples of suitable topical ocular administration include administration in eye drops and by spray formulations. A further suitable topical administration route is by subconjunctival injection. In the embodiments where the ophthalmic compositions are formulated into a dosage form selected from the group consisting of an eye drop, an eye wash, an eye spray, an ointment, a cream and a gel, it is recommended that the composition be topically applied to the eye once a day, twice a day or three times a day.

The ophthalmic composition is administered to the subject in a therapeutically effective amount to elicit a beneficial biological or medicinal response that is being sought in a cell, tissue, system, animal or human by a researcher, veterinarian, medical doctor or other clinician and preferably to stabilize, ameliorate, alleviate or relief myopia in the subject. For example, a therapeutically effective amount herein is a dose which leads to a clinically detectable improvement or treatment of the eye of a subject suffering from or at a risk for myopia. The clinically detectable improvement or treatment may be determined by evaluating refractive error (RE) with diopter measurements, or by measuring the expression of TNF-α, IL-6 or IL-8 in the afflicted eye. When administered to a human patient, the ophthalmic composition is preferably administered daily, weekly or twice a week, at an amount ranging from 0.01 ng/kg body weight/day to 100 ng/kg body weight/day, more preferably from 0.1 ng/kg/day to 10 ng/kg/day. Dose administration can be repeated depending upon the pharmacokinetic parameters of the dosage formulation and the route of administration used.

The following examples are given for the purpose of illustration only and are not intended to limit the scope of the invention. Statistical analyses were performed with SAS statistical software (version 9.4 for Windows; SAS Institute, Inc., Cary, N.C., USA). Statistical significance is defined as p<0.05.

### Example 1: Preparation of Aqueous Extract of P. cuspidatum

Rhizome slices of the herb were purchased from a local herbal vendor in Taichung City, Taiwan and ground into powder. 600 grams of the powder was weighted and soaked in 3,000 grams of double distilled water for 30 minutes. Extraction was conducted at 121° C. under a pressure of 1.2 kg/cm² for an hour. The resultant mixture was allowed to cool to ambient temperature and then centrifuged at 10,000 rpm for 30 minutes. The supernatant was collected and then filter sterilized with a 0.22 micron pore size filter. The filtrate thus obtained was aliquoted and stored at −20° C., which was abbreviated as Extract A in the following Examples. An aliquot of the extract was lyophilized to dryness on a rotary evaporator, and the concentration of the extract was determined to be 26.4 mg/ml by dividing the dry weight of the aliquot by the original volume of the aliquot before lyophilization.

### Example 2: Preparation of Aqueous Extract of P. vulgaris

The herb was purchased from a local herbal vendor in Taichung City, Taiwan and ground into powder. 600 grams of the powder was weighted and soaked in 5,500 grams of double distilled water for 30 minutes. Extraction was conducted at 121° C. under a pressure of 1.2 kg/cm² for an hour. The resultant mixture was allowed to cool to ambient temperature and then centrifuged at 10,000 rpm for 30 minutes. The supernatant was collected and then filter sterilized with a 0.22 micron pore size filter. The filtrate thus obtained was aliquoted and stored at −20° C., which was abbreviated as Extract C in the following Examples. An aliquot of the extract thus obtained was lyophilized to dryness on a rotary evaporator, and the concentration of the extract was determined to be 18.7 mg/ml by dividing the dry weight of the aliquot by the original volume of the aliquot before lyophilization.

### Example 3: Cytokine Combination for Inducing Inflammation

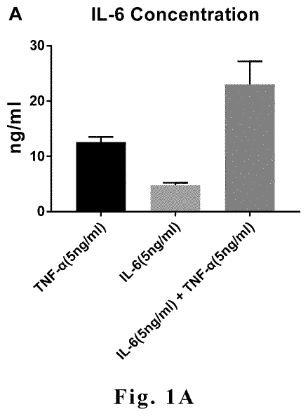

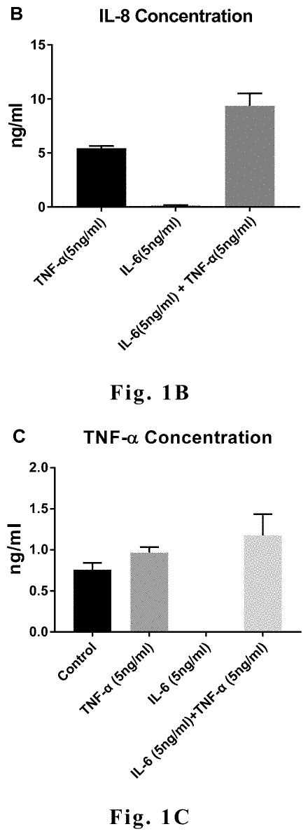

ARPE-19 human retinal pigment epithelial cells were purchased from the Bioresource Collection and Research Center, HsinChu City, Taiwan. Cells were inoculated in a 96-well microplate at a density of 1×10⁴ cells/well and cultured at 37° C. in a 5% CO₂ atmosphere in Dulbecco's Modified Eagle's medium (DMEM) (Life Technologies, Karlsruhe, Germany) supplemented with 10% fetal bovine serum (FBS) (Gibco®, Grand Island, N.Y., USA) and 1% antibiotic-antimycotic (Gibco®, Grand Island, N.Y., USA). ARPE-19 cells were treated as following: (1) Control group: no treatment; (2) IL-6 group: interleukin-6 (IL-6) at 5 ng/ml was added to the media; (3) TNF-α group: tumor necrosis factor-alpha (TNF-α) at 5 ng/ml was added to the media; (4) IL-6+TNF-α group: IL-6 at 5 ng/ml and TNF-α at 5 ng/ml were added to the media. The treatment lasted for 16 hours and the culture media in the respective groups were collected. The expressions of IL-6, IL-8 and TNF-α in the respective groups were measured using enzyme-linked immunosorbent assay (ELISA). The results are shown in FIGS. 1A-1C.

As shown in FIGS. 1A-1C, the combined administration of TNF-α and IL-6 significantly increased the expressions of IL-6, IL-8 and TNF-α in ARPE-19 cells at the protein level as compared with administration of either cytokine alone.

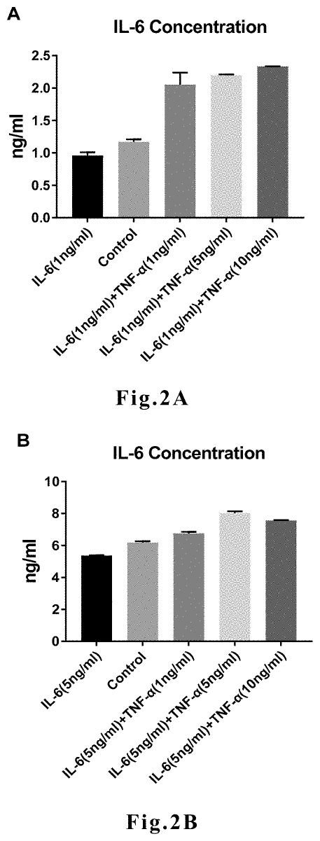

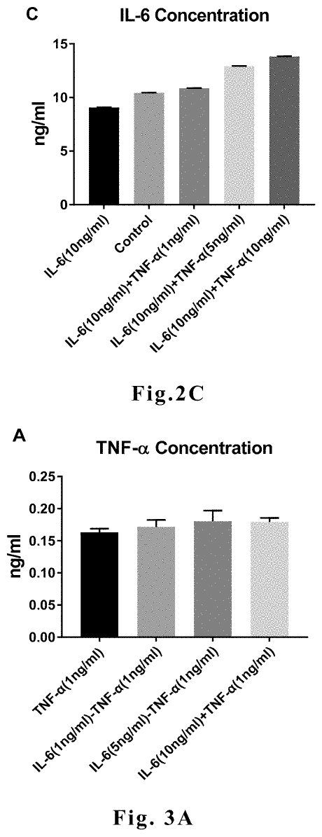

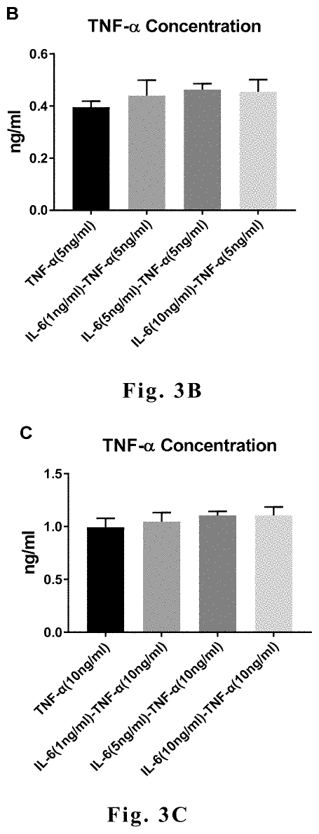

In a separate test, the experiment procedure described above was repeated, except that ARPE-19 cells were inoculated at a density of 3,000 cells/well and IL-6 was administered at 1, 5 or 10 ng/ml in combination with TNF-α at 1, 5 or 10 ng/ml. As shown in FIGS. 2A-2C and 3A-3C, similar results to those shown in FIGS. 1A-1C were obtained using IL-6 plus TNF-α at various concentrations. As such, 5 ng/ml IL-6 plus 5 ng/ml TNF-α were selected to induce inflammation in ARPE-19 cells in the following Examples.

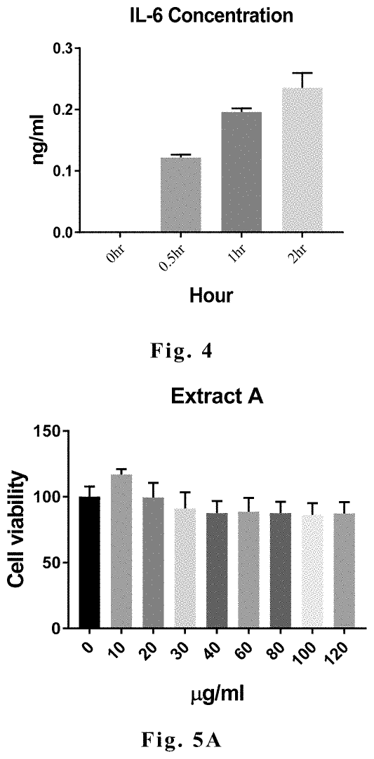

In a yet separate test, the experiment procedure was generally repeated in the absence or presence of 5 ng/ml IL-6 and 5 ng/ml TNF-α. The cytokine treatment lasted for 0.5, 1.0 or 2.0 hours, and then the culture media were removed. The ARPE-19 cells were washed twice with PBS and cultured in fresh DMEM supplemented with 10% FBS plus 1% antibiotic-antimycotic. The culture media in the respective groups were collected at the 6^{th} hour following the start of the cytokine treatment. The expressed levels of IL-6 in the respective wells were measured using ELISA. The results shown in FIG. 4 indicate that ARPE-19 cells kept secreting inflammatory mediators into the culture media after removal of IL-6 and TNF-α from the culture media.

### Example 4: Cytotoxicity Assay

ARPE-19 cells were inoculated in a 96-well microplate at a density of 3,000 cells/well and cultured overnight. The culture medium was refreshed, and the cells in the respective wells were incubated with Extract A or Extract C at various concentrations between 0-120 μg/ml for 72 hours. Afterwards, the extracts were removed and washed with phosphate buffered saline (PBS). MTS cytotoxicity assay was carried out to detect the cytotoxicity of the extracts to ARPE-19 cells, and the OD values were measured at 490 nm.

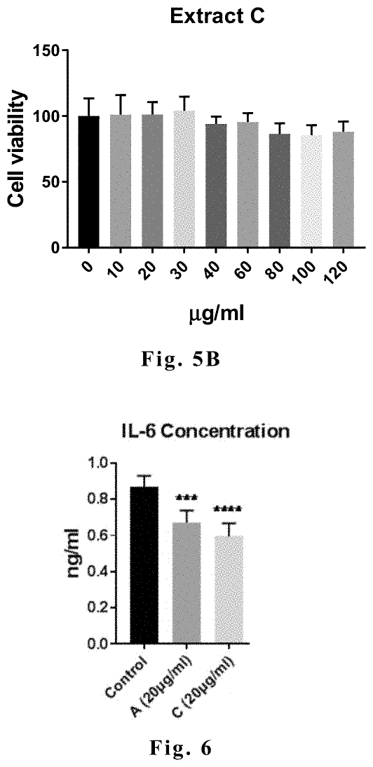

The results shown in FIGS. 5A-5B demonstrate that the viabilities of the ARPE-19 cells treated with the extract prepared in either Extract A or Extract C at the concentration ranging from 10 to 120 μg/ml were generally identical to those obtained from the untreated cells, indicating that neither Extract A nor Extract C was cytotoxic to ARPE-19 cells at the concentrations tested.

### Example 5: Individual Inhibitory Effects of Extract A and Extract C on Inflammation

ARPE-19 cells were inoculated in a 96-well microplate at a density of 1×10⁴ cells/well and cultured for 2 hours in the presence of 5 ng/ml IL-6 and 5 ng/ml TNF-α to induce inflammation. Then, the culture media were replaced with fresh DMEM supplemented with 10% FBS plus 1% antibiotic-antimycotic. The cell cultures in the respective wells were added with culture medium (as the Control), 20 μg/ml Extract A or 20 μg/ml Extract C and incubated for additional 6 hours. The culture media in the respective groups were collected, and the expressed levels of IL-6 in the respective wells were measured using ELISA. As shown in FIG. 6, IL-6 was expressed at a level of 0.86±0.06 ng/ml in the Control group (treated with culture medium only), whereas IL-6 was at the levels of 0.67±0.06 ng/ml and 0.59±0.07 ng/ml in the group of treated with Extract A and Extract C, respectively. The results indicate that Extract A and Extract C both exhibited suppressive effects on the cytokine-induced inflammation in ARPE-19 cells.

### Example 6: Combined Inhibitory Effects of Extract A and Extract C on Cytokine-Induced Inflammation

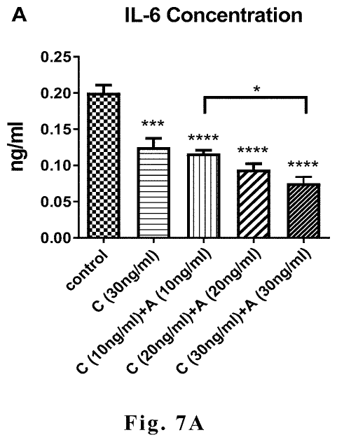

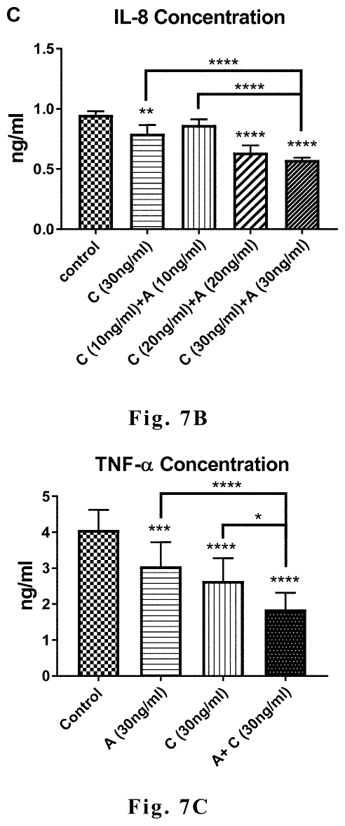

ARPE-19 cells were inoculated in a 96-well microplate at a density of 1×10⁴ cells/well and cultured for 2 hours in the presence of 5 ng/ml IL-6 and 5 ng/ml TNF-α to induce inflammation. Then, the culture media were replaced with fresh DMEM supplemented with 10% FBS plus 1% antibiotic-antimycotic. The cell cultures in the respective wells were incubated for additional 6 hours with culture medium (as the Control), Extract C alone, or a combination of Extract A with Extract C at various concentrations between 10 ng/ml to 30 ng/ml. The culture media in the respective groups were collected, and the expressed levels of IL-6 and IL-8 in the respective wells were measured using ELISA. As shown in FIGS. 7A and 7B, the expressions of IL-6 and IL-8 were both suppressed by the herb extract combination of Extract A with Extract C in a dose-dependent manner, and the herb extract combination, when Extract A and Extract C were at a concentration of no less than 20 ng/ml, exhibited significantly higher inhibitory effects as compared with the Control and Extract C alone (p<0.05).

In a separate test, the experiment procedure stated above was repeated, except that the cell cultures in the respective wells were treated with culture medium (as the Control), Extract A alone, Extract C alone, or a combination of Extract A with Extract C, and that the expression of TNF-α was measured. As shown in FIG. 7C, the herb extract combination of Extract A with Extract C exhibited significantly higher inhibitory effects on the expression of TNF-α as compared with the Control, Extract A alone, and Extract C alone (p<0.05).

The results herein demonstrate that Extract A and Extract C exhibited synergistic inhibitory effects on the cytokine-induced inflammation in ARPE-19 cells.

### Example 7: Combined Inhibitory Effects of Extract A and Extract C on Cytokine-Induced Inflammation

Example 6 was generally repeated, except that the inhibitory effects of the herb extract combination was tested on human retinal pigment epithelial (RPE) primary cells. The human RPE primary cells used herein were purchased from ScienCell Research Laboratories, Carlsbad, Calif., USA (Catalog #6540).

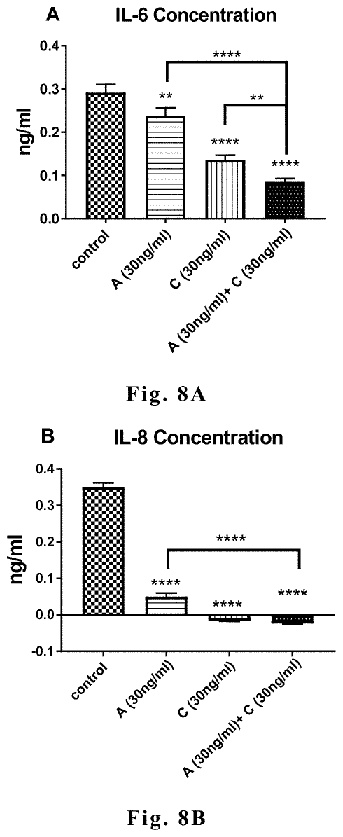

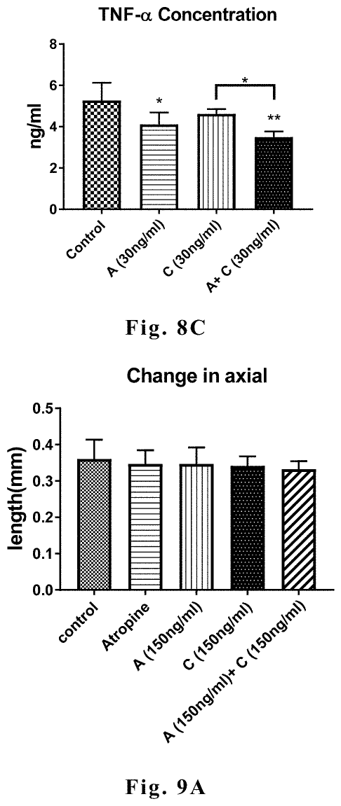

As shown in FIGS. 8A-8C, similar results to those shown in FIGS. 7A-7C were obtained using human RPE primary cells, where the herb extract combination of Extract A with Extract C exhibited higher inhibitory effects on the expression of TNF-α as compared with the Control, Extract A alone, and Extract C alone, indicating that Extract A and Extract C exhibited synergistic inhibitory effects on the cytokine-induced inflammation in human RPE primary cells.

### Example 8: Preparation of Eye Drops

0.125% atropine eye drop was prepared under sterile conditions by dilution of 1% atropine ophthalmic solution with artificial tears (commercially available from Alcon Laboratories, Inc., Fort Worth, Tex., USA). The Extract A and/or Extract C were mixed with 5 ml artificial tears (commercially available from Alcon Laboratories, Inc., Fort Worth, Tex., USA) to reach a final concentration of 150 ng/ml by an oscillator.

### Example 9: MFD Animal Model

3-week old Golden Syrian hamsters, each weighing 80 to 90 grams, were used for experiments. All animals were kept in a 12-hour light/dark cycle. All procedures were approved by the Institutional Animal Care and Use Committee of China Medical University and were conducted in accordance with the guidelines of the Use of Animals in Ophthalmic and Vision Research. Hamsters were raised with right eyelid fusion for 21 days. MFD was induced in the right eyes of the animals, with their left eyes serving as a control. The animals were randomly assigned to treatment or control groups receiving daily applications of artificial tears or the eye drops prepared in Example 8, respectively, to both eyes. The treatment lasted for 21 days. The axial lengths of both eyes in each animal were measured on the 21^{st} day following the start of the treatment by A-scan ultrasonography (PacScan 300 Plus, NY, USA).

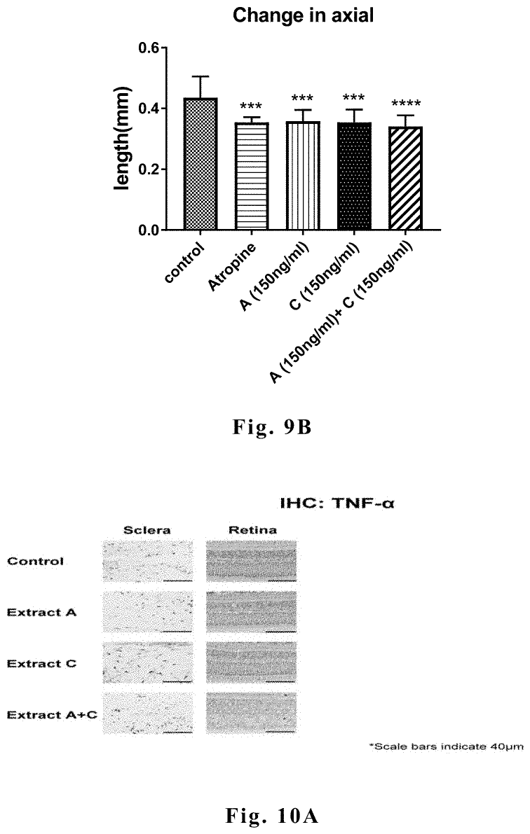

As shown in FIG. 9A, the axial lengths of the non-MFD (left) eyes after a 21-day treatment were 0.36±0.05 mm (Control, treated with artificial tears only), 0.33±0.03 mm (treated with Atropine) and 0.33-0.34 mm (treated with herb extracts), the differences among which are not statistically significant. As shown in FIG. 9B, the axial lengths of the MFD (right) eyes in the treatment groups were 0.358±0.01 mm (treated with Atropine), 0.35±0.03 mm (treated with 150 ng/ml Extract A), 0.354±0.04 mm (treated with 150 ng/ml Extract C), and 0.34±0.03 mm (treated with 150 ng/ml Extract A plus 150 ng/ml Extract C), respectively, all of which were significantly lower as compared with the results in animals receiving artificial tears only (0.436±0.06 mm in the Control group) (p<0.05). The data herein suggest that administration of the herb extracts inhibited myopia progression.

### Example 10: Immunohistochemical (IHC) Analysis

The animals described in Example 9 were sacrificed, and eyes were collected from the treatment and control groups, embedded in paraffin, and cut at a thickness of 20 μm. Sections were collected on glass slides. Antigen retrieval was performed by boiling the slides in citrate buffer (pH 6.0), and sections were then stained with antibodies against IL-6, IL-8, TNF-α, NFκB, MMP2 and CD55. The EnVision System peroxidase kit (DAKO, Carpentaria, Calif., USA) was used to visualize immunoreactivity.

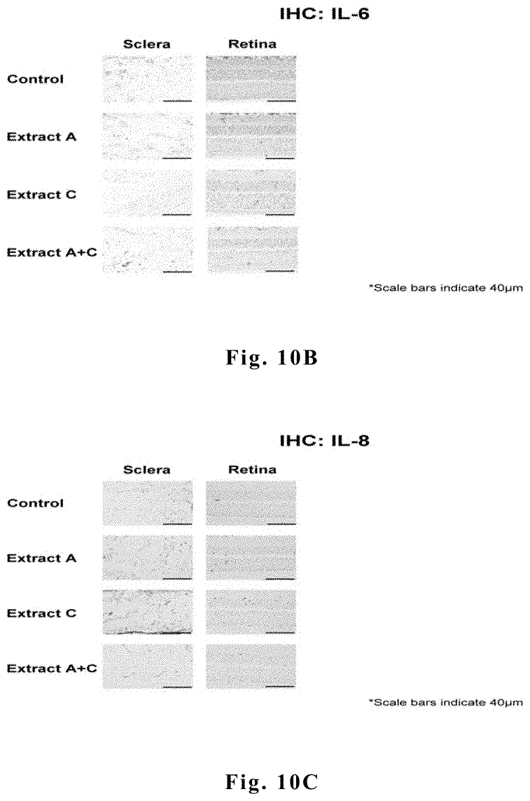

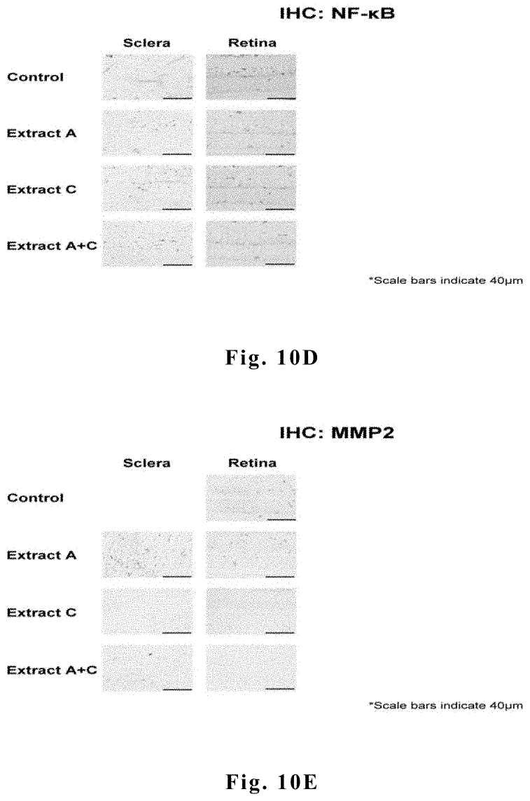

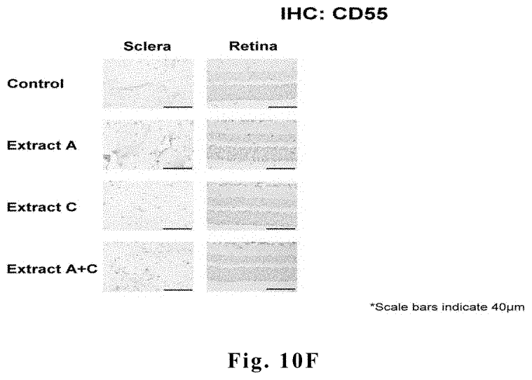

As shown in FIGS. 10A-10F, the expression of inflammatory factors TNF-α, IL-6, IL-8, NFκB and MMP2 in hamster retina tissues in the treatment groups was were significantly lower as compared with that observed in animals receiving artificial tears only, while the expression of an inflammatory suppressor, i.e., decay accelerating factor (CD55), in the treatment groups was higher than the control group. The results revealed that inflammation was suppressed in the treatment groups, consistent with the results obtained in Example 9.

While the invention has been described with reference to the preferred embodiments above, it should be recognized that the preferred embodiments are given for the purpose of illustration only and are not intended to limit the scope of the present invention and that various modifications and changes, which will be apparent to those skilled in the relevant art, may be made without departing from the spirit and scope of the invention.

All papers, publications, literature, patents, patent applications, websites, and other printed or electronic documents referred herein, including but not limited to the references listed below, are incorporated by reference in their entirety. In case of conflict, the present description, including definitions, will prevail.

We claim:

1. An ophthalmic composition comprising a therapeutically effective amount of an aqueous extract derived from a botanical species selected from the group consisting of Polygonum cuspidatum Sieb. et Zucc., Prunella vulgaris, and a combination thereof, in combination with an ophthalmically acceptable carrier.

2. The composition according to claim 1, which consists essentially of a therapeutically effective amount of the aqueous extract derived from a botanical species selected from the group consisting of Polygonum cuspidatum Sieb. et Zucc., Prunella vulgaris, and a combination thereof, in combination with an ophthalmically acceptable carrier.

3. The composition according to claim 2, which consists of a therapeutically effective amount of the aqueous extract derived from a botanical species selected from the group consisting of Polygonum cuspidatum Sieb. et Zucc., Prunella vulgaris, and a combination thereof, in combination with an ophthalmically acceptable carrier.

4. The composition according to claim 1, wherein the aqueous extract is obtained using an aqueous solvent selected from water, C₁₋₆ alkanols, C₁₋₄ alkylene glycols, aqueous carbohydrate solutions, salt solutions thereof, and mixtures thereof.

5. The composition according to claim 4, wherein the aqueous solvent is selected from water and salt solutions thereof.

6. The composition according to claim 5, wherein the ophthalmic composition is formulated into a dosage form selected from the group consisting of an eye drop, an eye wash, an eye spray, an ointment, a cream and a gel.

7. The composition according to claim 6, wherein the ophthamically acceptable carrier is selected from the group consisting of polyoxyethylene castor oil ether (Cremophor EL), alkyl dimethyl benzyl ammonium (BKC), lecithin, cholesterol, phosphate-buffered saline (PBS), cyclodextrin, tween 80, castor oil, artificial tears, and a combination thereof.

8. The composition according to claim 7, wherein the ophthalmically acceptable carrier comprises artificial tears.

9. A method for treating myopia in a subject, comprising administering to said subject an ophthalmic composition comprising a therapeutically effective amount of an aqueous extract derived from a botanical species selected from the group consisting of Polygonum cuspidatum Sieb. et Zucc., Prunella vulgaris, and a combination thereof, in combination with an ophthalmically acceptable carrier.

10. The method according to claim 9, wherein the ophthalmic composition consists essentially of a therapeutically effective amount of the aqueous extract derived from a botanical species selected from the group consisting of Polygonum cuspidatum Sieb. et Zucc., Prunella vulgaris, and a combination thereof, in combination with an ophthalmically acceptable carrier.

11. The method according to claim 10, wherein the ophthalmic composition consists of a therapeutically effective amount of the aqueous extract derived from a botanical species selected from the group consisting of Polygonum cuspidatum Sieb. et Zucc., Prunella vulgaris, and a combination thereof, in combination with an ophthalmically acceptable carrier.

12. The method according to claim 9, wherein the aqueous extract is obtained using an aqueous solvent selected from water, C₁₋₆ alkanols, C₁₋₄ alkylene glycols, aqueous carbohydrate solutions, salt solutions thereof, and mixtures thereof.

13. The method according to claim 12, wherein the aqueous solvent is selected from water and salt solutions thereof.

14. The method according to claim 13, wherein the ophthalmic composition is formulated into a dosage form selected from the group consisting of an eye drop, an eye wash, an eye spray, an ointment, a cream and a gel.

15. The method according to claim 14, wherein the ophthamically acceptable carrier is selected from the group consisting of polyoxyethylene castor oil ether (Cremophor EL), alkyl dimethyl benzyl ammonium (BKC), lecithin, cholesterol, phosphate-buffered saline (PBS), cyclodextrin, tween 80, castor oil, artificial tears, and a combination thereof.

16. The method according to claim 15, wherein the ophthalmically acceptable carrier comprises artificial tears.

17. The method according to claim 16, wherein the subject is selected from the group consisting of human and non-human vertebrates.

18. The method according to claim 17, wherein the subject is human.

19. The method according to claim 18, wherein the ophthalmic composition is administered at an amount ranging from 0.01 ng/kg body weight/day to 100 ng/kg body weight/day.

20. The method according to claim 19, wherein the ophthalmic composition is administered at an amount ranging from 0.1 ng/kg body weight/day to 10 ng/kg body weight/day.
